(19) United States
(12) Patent Application Publication
(10) Pub. No.: US 2007/0053480 A1
(43) Pub. Date: Mar. 8, 2007

(54) X-ray CT apparatus

(75) Inventors: Akihiko Nishide, Tokyo (JP); Tetsuya Horiuchi, Tokyo (JP); Yasuhiro Imai, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/507,763

(22) Filed: Aug. 22, 2006

(30) Foreign Application Priority Data
Aug. 25, 2005 (JP) 2005-244107

(51) Int. Cl. H05G 1/60; A61B 6/00; G01N 23/00; G21K 1/12

(52) U.S. Cl. 378/4

(57) ABSTRACT

This invention provides an X-ray CT capable of presenting information of exposure to the operator by displaying X-ray dose information of each of regions of interest to be scanned by an X-ray CT apparatus, thereby encouraging reduction in exposure and optimization. X-ray dose information of each region to be scanned by a conventional scan (axial scan), a cine scan, a helical scan, or a variable-pitch helical scan of an X-ray CT apparatus is displayed so that the operator can recognize the X-ray dose information before acquisition of an image of a subject. The X-ray dose information can be predicated with higher precision and displayed by using a dose prediction value obtained by an interpolation value and an extrapolation value of the first or higher order on at least three or more kinds of phantom measurement values, not a simple prediction value such as a zero-th order interpolation value or a zero-th order extrapolation value obtained by using measurement values of two kinds of phantoms like in the present CTDI display.

### CROSS REFERENCE TO RELATED APPLICATIONS

This application claims the benefit of Japanese Application No. 2005-244107 filed Aug. 25, 2005.

### BACKGROUND OF THE INVENTION

The present invention relates to an X-ray CT (Computed Tomography) image acquiring method using an X-ray CT apparatus for medical or industrial use, and to an X-ray CT apparatus. More particularly, the invention relates to a display method of displaying X-ray dose information of each region of interest in a conventional scan (axial scan in other word), a cine scan, a helical scan, or a variable-pitch helical scan to an operator, thereby encouraging reduction in exposure and optimization.

Conventionally, in an X-ray CT apparatus using a multi-row X-ray detector or an X-ray CT apparatus using a matrix structure two-dimensional X-ray area detector typified by a flat panel, in the case of acquiring images from the neck to the liver or from the lung field to the liver by a helical scan as shown in FIG. 12, at the time of setting image acquisition parameters by image acquisition parameter setting device, X-ray dose information such as a CTDI (Computed Tomography Dose Index) value and a DLP (Dose-Length Product) value in the case of acquiring images from the neck to the liver or from the lung field to the liver is displayed. The CTDI value indicates X-ray dose of one scan, and the DLP indicates X-ray dose of one test (refer to, for example, Japanese Patent Laid-Open No. 2005-74000 (pp. 7 to 9 and FIGS. 3 to 9).

In the case of acquiring images of the head by a conventional scan (axial scan) or a cine scan as shown in FIG. 13, if images are acquired by performing the conventional scan (axial scan) or cine scan a plurality of times in a plurality of positions in the z direction, X-ray dose information such as CTDI value, DLP value, or the like of a single conventional scan (axial scan) or cine scan, or of the whole conventional scan (axial scan) or cine scan in the plurality of positions in the z direction, is displayed.

Consequently, also in a helical scan, only X-ray dose information of a part in the image acquisition range in the z direction subjected to a conventional scan (axial scan) or a cine scan, for example, an image acquisition range in the z direction of a part corresponding to the region of interest corresponding to a part of an organ cannot be known directly on a display screen.

The conventional method has the problem from the viewpoint of directly displaying X-ray dose information of only the region of interest.

A CTDI value is obtained by weighted addition on X-ray dose values in the center portion and the peripheral portions in two acrylic cylindrical phantoms and determined every field of view of image acquisition as shown in FIG. 16. A value D_{CTDI16} obtained by performing weighted addition on an X-ray dose value D_{CTDI16C} in the center portion and an X-ray dose value D_{CTDI16P} in the peripheral portions in an acrylic 16-cm circular cylinder is calculated as follows.

$${D}_{CTDI 16} = \frac{1}{3} {D}_{CTDI 16 C} + \frac{2}{3} {D}_{CTDI 16 P} \qquad Equation 1$$

A value D_{CTDI32} obtained by performing weighted addition on an X-ray dose value D_{CTDI32C} in the center portion and an X-ray dose value D_{CTDI32P} in the peripheral portion in an acrylic 32-cm circular cylinder as shown in FIG. 15 is calculated as follows.

$${D}_{CTDI 32} = \frac{1}{3} {D}_{CTDI 32 C} + \frac{2}{3} {D}_{CTDI 32 P} \qquad Equation 2$$

D_{CTDI16C} is an X-ray dose value in a center position A of a phantom in FIG. 14.

D_{CTDI16P} is an average value of X-ray dose values in eight peripheral positions B to I of the phantom in FIG. 14.

Similarly, D_{CTDI32C} is an X-ray dose value in a center position A of a phantom in FIG. 15.

D_{CTDI32P} is an average value of X-ray dose values in eight peripheral positions B to I of the phantom in FIG. 15.

In FIG. 16, the CTDI value is determined depending only on the size of the field of view and the diameter of the field of view of image acquisition which is set by image acquisition parameter setting device. In this case, there are the following problems.

1. No influence is exerted by the size of a subject.

2. The CTDI values in the fields of view of image acquisition are determined by 0-th order interpolation and 0-th order extrapolation on CTDI values of two acrylic circular cylinders.

Since a DLP (Dose Length Product) value is an integrated value in the z direction of the CTDI values, there are problems similar to the above.

As described above, the CTDI value and the DLP value are not influenced by the size of a subject and are not proportional to the field of view of image acquisition, so that the operator cannot correctly grasp a value of X-ray dose exposure of the subject. Consequently, in the case where the operator increases the X-ray dose so that the picture quality of a tomographic image of the subject does not deteriorate, the operator may not know that he/she sets image acquisition parameters with which the subject is exposed to an X-ray of an extra dose. Due to this, there is the possibility that exposure of the subject becomes excessive if X-ray dose information such as a CTDI value and a DLP value is not correctly displayed, and this is a problem from the viewpoint of X-ray exposure.

On the other hand, in an X-ray CT apparatus using a multi-row X-ray detector or an X-ray CT apparatus using a matrix structure two-dimensional X-ray area detector typified by a flat panel, the thickness in the z direction of a tomographic image captured is decreasing and the size of pixels in an XY plane as a tomographic image plane is decreasing. In the case where the operator tries to have higher picture quality of a thin tomographic image, the possibility that a dose of an X-ray applied to the subject tends to be excessive is high. Consequently, only X-ray dose information based on more accurate size of a subject or, considering variations in the sensitivity to a damage caused by X-rays among regions of the subject, only the X-ray dose information of a series of image acquisition of a helical scan, a conventional scan (axial scan), or a cine scan may be too rough as the X-ray dose information in future.

### SUMMARY OF THE INVENTION

Therefore, an first object of the present invention is to provide an X-ray CT apparatus capable of providing X-ray dose information in finer unit for each region of interest or the like in a subject while executing an image acquisition parameters setting process of particularly a conventional scan (axial scan), a cine scan, a helical scan, or a variable-pitch helical scan of an X-ray CT apparatus using a X-ray detector such as multi-row X-ray detector or a matrix structure two-dimensional X-ray area detector typified by a flat panel.

Further object of the present invention is to provide an X-ray CT apparatus capable of providing more-accurate X-ray dose information based on the size of a subject while executing an image acquisition parameters setting process of particularly a conventional scan (axial scan), a cine scan, a helical scan, or a variable-pitch helical scan of an X-ray CT apparatus using a X-ray detector such as multi-row X-ray detector or a matrix structure two-dimensional X-ray area detector typified by a flat panel.

The present invention can provide X-ray dose information based on a finer unit. Further, the present invention can provide of more-accurate X-ray dose information on the basis of the size of a subject by using the profile area of the subject obtained from a scout view and the like. The invention solves the problem by providing an X-ray CT apparatus characterized in that it can provide more-accurate X-ray dose information based on a finer unit of a region of interest of the subject determined on a scout view.

According to a first aspect, the present invention provides an X-ray CT apparatus including: a device for acquiring projection data of an X-ray passed through a subject positioned between an X-ray generator and an X-ray detector which are opposite to each other; a device for reconstructing an image from the projection data acquired by said device for acquiring the projection data; a device for displaying a tomographic image obtained by said device for reconstructing the image; a setting device for setting various image acquisition parameters for acquisition of a tomographic image; and a device for displaying X-ray dose information of a partial region of an image acquisition region provided by one scan when an image acquisition parameter setting process is executed.

The X-ray CT apparatus according to the first aspect, X-ray dose information in a finer unit of a subject can be provided. For example, X-ray dose information of an image acquisition region in a z direction as part of a series of z-direction image acquisition ranges in a helical scan, a variable-pitch helical scan, a conventional scan (axial scan), or a cine scan can be provided.

According to a second aspect, the X-ray CT apparatus according to the first aspect is characterized in that said X-ray detector is any one of a matrix structure two-dimensional X-ray area detector, a flat panel X-ray detector, and a multi X-ray detector.

The X-ray CT apparatus according to the second aspect, which uses an X-ray detector is one selected from a matrix structure two-dimensional X-ray area detector, X-ray dose information in a finer unit of a subject can be provided. For example, X-ray dose information of an image acquisition region in a z direction as part of a series of z-direction image acquisition ranges in a helical scan, a variable-pitch helical scan, a conventional scan (axial scan), or a cine scan can be provided.

In a third aspect of the present invention, the X-ray CT apparatus according to the first is characterized in that said device for displaying the X-ray dose information includes a device for displaying X-ray dose information of a partial region of an image acquisition region provided by one scan in a z-direction as a direction of a body axis of the subject when an image acquisition parameter setting process of a conventional scan or an axial scan is executed.

According to the third aspect, the X-ray CT apparatus can provide X-ray dose information in unit of tomographic images as a part of a plurality of tomographic images acquired by a single conventional scan (axial scan), that is, X-ray dose information of a part of a series of z-direction image acquisition ranges, so that can provide X-ray dose information in a finer unit of a subject.

According to a fourth aspect, the invention provides an X-ray CT apparatus according to the first aspect is characterized in that said device for displaying the X-ray dose information includes a device for displaying X-ray dose information of a partial region of an image acquisition region provided by one scan in a z-direction as a direction of a body axis of the subject when an image acquisition parameter setting process of a helical scan or a variable-pitch helical scan is executed.

According to the fourth aspect, the X-ray CT apparatus can provide X-ray dose information in a unit of tomographic images as a part of a plurality of tomographic images obtained by a single helical scan, that is, in a part of a series of z-direction image acquisition ranges. Thus, X-ray dose information in a finer unit of a subject can be provided.

According to a fifth aspect of the invention, the X-ray CT apparatus according to the first aspect is characterized in that said device for displaying the X-ray dose information includes a device for displaying X-ray dose information of a partial region of an image acquisition region provided by one scan in a z-direction as a direction of a body axis of the subject or in a time direction when an image acquisition parameter setting process of a cine scan is executed.

According to the fifth aspect, the X-ray CT apparatus can provide X-ray dose information in a unit of tomographic images as a part of a plurality of tomographic images obtained by a single helical scan, that is, in a part of a series of z-direction image acquisition ranges. Thus, X-ray dose information in a finer unit of a subject can be provided. Since a single cine scan is performed in a time range, X-ray dose information based on a further finer unit in a part of the time range can be also provided.

According to a sixth aspect of the invention, an X-ray CT apparatus according to the first aspect is characterized in that said partial region is being set on a scout view of the subject.

In the X-ray CT apparatus according to the sixth aspect, a partial region such as region of interest is preliminarily set on a scout view. At the time of setting image acquisition parameters by the image acquisition parameter setting device, dose information of an X-ray applied to the region of interest is displayed and presented to the operator. Consequently, X-ray dose information in a finer unit can be provided.

In a seventh aspect of the invention, the X-ray CT apparatus according to the first aspect is characterized in that said partial region is a region of interest and being set by setting a part of one scan range in a z-direction and, in a case where a vertical direction perpendicular to the z-direction is set as a y-direction and a direction perpendicular to the z-direction and the y-direction is set as an x-direction, designating a range in at least one of the x-direction and the y-direction.

In the X-ray CT apparatus according to the seventh aspect, a region of interest is set by designating an image acquisition range in the z direction and an image acquisition range in the x and y directions on a scout view, so that X-ray dose information corresponding to the region of interest in the cross section of the subject is obtained. Thus, X-ray dose information in a finer unit based on the size of the subject can be provided.

In an eighth aspect of the present invention, the X-ray CT apparatus according to the first aspect is characterized in that wherein said X-ray dose information includes at least one of a CTDI value, a DLP value, and efficiency for X-ray utilization.

In the X-ray CT apparatus according to the eighth aspect, generally, a CTDI value, a DLP value, and the like are known as X-ray dose information. From the CTDI value, the DLP value, and the like, the operator can predict dose of an X-ray applied to the subject, estimate a damage of the subject caused by the X-ray, and evaluate the adequacy of the X-ray dose.

In a ninth aspect of the invention, the X-ray CT apparatus according to the first aspects is characterized in that said X-ray dose information includes a value depending on a sectional area of the subject or an X-ray profile area obtained from a scout view of the subject.

In the X-ray CT apparatus according to the ninth aspect, a damage caused by the X-ray on the subject depends on the sectional area of the subject. Consequently, by obtaining dose information of an X-ray applied to the subject from the sectional area of the subject or the X-ray profile area, more-accurate X-ray dose information based on the size of a subject can be obtained.

According to a tenth aspect of the invention, the X-ray CT apparatus according to the ninth aspect is characterized in that said sectional area is predicted from at least one of height, weight, age, an image acquisition part, and sex of the subject.

The X-ray CT apparatus according to the tenth aspect can statistically predict the sectional area of a subject to some extent by using height, weight, age, a region of image acquisition, and sex. The dose information of an X-ray applied to the subject can be predicted from the predicted sectional area of the subject.

According to an eleventh aspect, the X-ray CT apparatus according to the ninth aspect is characterized in that said sectional area is predicted from the X-ray profile.

The X-ray CT apparatus according to the eleventh aspect, the X-ray profile area of the subject can be obtained from a scout view. Thus, dose information of an X-ray applied to the subject can be obtained from the X-ray profile image obtained from the scout view.

### EFFECTS OF THE INVENTION

According to the X-ray CT apparatus or the X-ray CT image reconstructing method of the present invention, the X-ray CT apparatus capable of providing more-accurate X-ray dose information based on the size of a subject and more-accurate X-ray dose information in finer unit for each region of interest in a subject which is set at the time of setting image acquisition parameters in a conventional scan (axial scan), a cine scan, a helical scan, or a variable-pitch helical scan of an X-ray CT apparatus having a multi-row X-ray detector or a two-dimensional area sensor of a matrix structure typified by a flat panel X-ray detector can be realized.

### BRIEF DESCRIPTION OF THE DRAWINGS

FIG. 1 is a block diagram showing an X-ray CT apparatus according to an embodiment of the present invention.

FIG. 2 is a diagram illustrating rotation of an X-ray generator (X-ray tube) and a multi-row X-ray detector.

FIG. 3 is a flowchart showing schematic operations of the X-ray CT apparatus according to the embodiment of the invention.

FIG. 4 is a flowchart showing the details of pre-process.

FIG. 5 is a flowchart showing the details of a three-dimensional image reconstructing process.

FIGS. 6a and 6b are conceptual diagrams showing a state where lines on a reconstruction region are projected in an X-ray transmission direction.

FIG. 7 is a conceptual diagram showing lines projected on a detector surface.

FIG. 8 is a conceptual diagram showing a state where projection data Dr (view, x, y) is projected onto a reconstruction region.

FIG. 9 is a conceptual diagram showing back projection pixel data D2 of each of pixels on the reconstruction region.

FIG. 10 is a diagram illustrating a state of obtaining back projection data D3 by adding the back projection pixel data D2 of the whole view in a pixel correspondence manner.

FIGS. 11a and 11b are conceptual diagrams showing a state where lines on a circular reconstruction region are projected in the X-ray transmission direction.

FIG. 12 is a diagram showing a helical scan from a lung field to the liver (a).

FIG. 13 is a diagram showing an axial scan of the head (b).

FIG. 14 is a diagram showing X-ray dose measurement positions in the center and the peripheral portions of an acrylic 16-cm circular cylinder.

FIG. 15 is a diagram showing X-ray dose measurement positions in the center and the peripheral portions of an acrylic 32-cm circular cylinder.

FIG. 16 is a diagram showing CTDI values according to the diameters of field of view of image acquisition.

FIG. 17 is a flowchart showing the flow of acquiring images of a subject.

FIG. 18 is a diagram showing a region of interest which is set on a scout view in the 90-degree direction.

FIG. 19 is a diagram showing a region of interest which is set on a scout view in the 0-degree direction.

FIG. 20 is a diagram showing examples of X-ray water substitute phantoms of various diameters.

FIG. 21 is a flowchart for obtaining X-ray dose information of a subject from a profile area.

FIG. 22 is a diagram showing linear approximation of a CTDI value.

FIG. 23 is a diagram showing a three-dimensional region of interest in continuous tomographic images of a subject.

FIG. 24 is a diagram showing a three-dimensional region of interest in continuous tomographic images of a subject.

FIG. 25 is a diagram showing a three-dimensional region of interest in continuous tomographic images of a subject.

FIG. 26 is a diagram showing correspondence between a set region of interest and a phantom on the basis of a sectional area of a subject.

FIG. 27 is a diagram showing a helical scan from the lung field to liver.

FIG. 28 is a diagram showing an axial scan of the head.

FIGS. 29a, 29b, 29c, and 29d are diagrams showing the case of a variable-pitch helical scan.

FIGS. 30a and 30b showing height, weight, a sectional area of a region, and a sectional area of a water substitute acrylic phantom.

### DETAILED DESCRIPTION OF THE INVENTION

The present invention will be described in more detail hereinbelow by embodiments shown in the drawings. However, the invention is not limited by the embodiments.

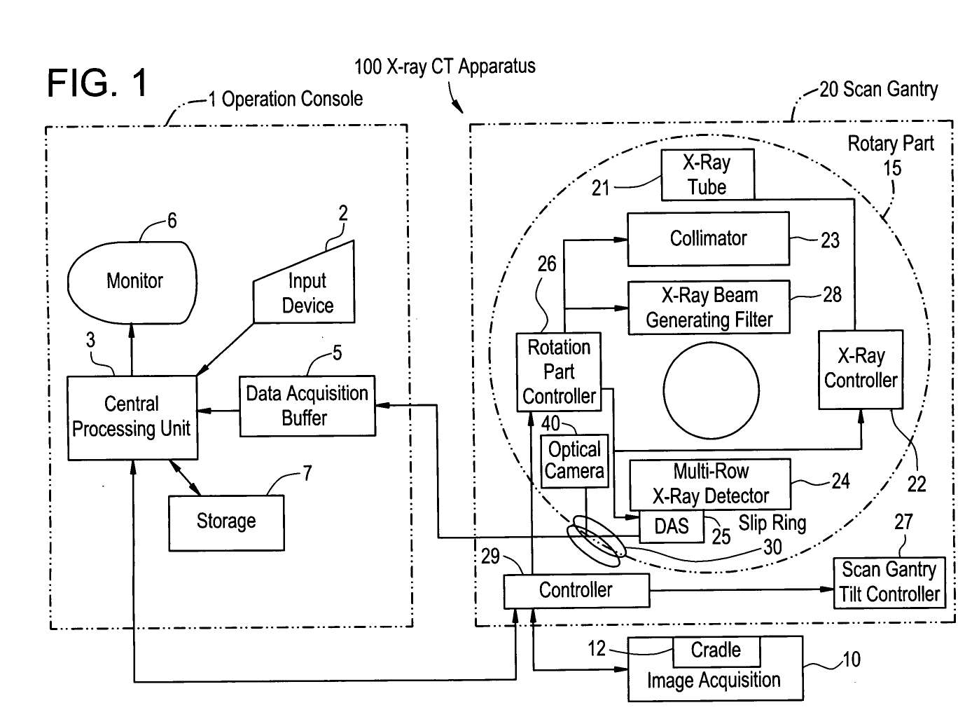

FIG. 1 is a configuration block diagram of an X-ray CT apparatus according to an embodiment of the present invention. An X-ray CT apparatus 100 has an operation console 1, an image acquisition table 10, and a scan gantry 20.

The operation console has an input device 2 for receiving an input of the operator, a central processing unit 3 for executing pre-process, image reconstructing process, post-process, and the like, a data acquisition buffer 5 for acquiring X-ray detector data obtained by the scan gantry 20, a monitor 6 for displaying a tomographic image obtained by reconstructing projection data obtained by pre-processing the X-ray detector data, and a storage 7 for storing a program, the X-ray detector data, the projection data, and an X-ray tomographic image.

The image acquisition parameters are input to the input device 2 and stored in the storage 7.

The image acquisition table 10 has a cradle 12 on which a subject is mounted and which is loaded/unloaded to/from an opening of the scan gantry 20. The cradle 12 is moved vertically and linearly by a motor built in the image acquisition table 10.

The scan gantry 20 has an X-ray tube 21, an X-ray controller 22, a collimator 23, an X-ray beam generating filter 28, a multi-row X-ray detector 24, a DAS (Data Acquisition System) 25, a rotary part controller 26 for controlling the X-ray tube 21 and the like rotating around the body axis of a subject, and a controller 29 for transmitting/receiving a control signal and the like to/from the operation console 1 and the image acquisition table 10. The X-ray beam generating filter 28 is an X-ray filter whose thickness is the smallest in the direction of an X-ray traveling to the center of rotation as a center of image acquisition and increases toward the periphery, so that a larger amount of an X-ray can be absorbed. Consequently, the exposure of the body surface of a subject whose section has a shape close to a circular shape or an elliptical shape can be reduced. The scan gantry 20 can be tilted forward and backward in the z direction by about ±30 degrees by a scan gantry tilt controller 27.

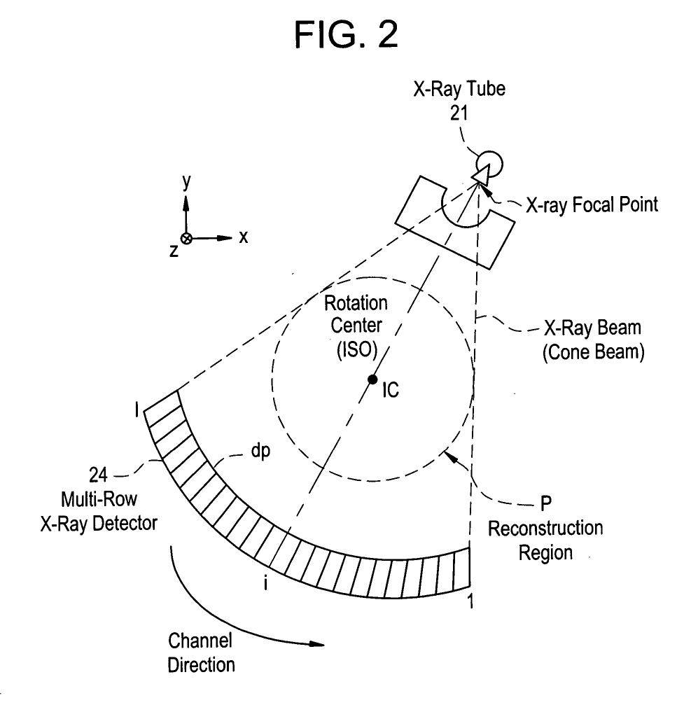

FIG. 2 is a diagram illustrating geometrical layout of the X-ray tube 21 and the multi-row X-ray detector 24.

The X-ray tube 21 and the multi-row X-ray detector 24 revolve around the rotation center IC. When the vertical direction is set as the y direction, the horizontal direction is set as the x direction, and the table travel direction perpendicular to the y and x directions is set as the z direction, the rotation plane of the X-ray tube 21 and the multi-row X-ray detector 24 is the xy plane. The travel direction of the cradle 12 is the z direction.

The X-ray tube 21 generates an X-ray beam called a cone beam CB. When the direction of the center axis of the cone beam CB is parallel to the y direction, the view angle is zero.

The multi-row X-ray detector 24 has, for example, 256 X-ray detector rows. Each X-ray detector row has, for example, 1,024 X-ray detector channels.

Projection data acquired from X-ray radiation is sent from the multi-row X-ray detector 24 and A/D converted by the DAS 25. The resultant digital data is supplied to the data acquisition buffer 5 via a slip ring 30. The data input to the data acquisition buffer 5 is processed by the central processing unit 3 in accordance with a program in the storage 7 and reconstructed to a tomographic image, and the tomographic image is displayed on the monitor 6.

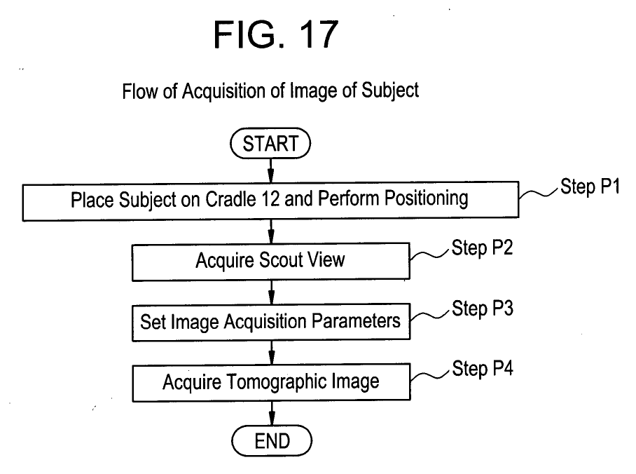

FIG. 17 is a flowchart showing an outline of operations of the X-ray CT apparatus of the embodiment.

In step P1, the subject is placed on the cradle 12 and positioning is performed. A slice write center position of the scan gantry 20 is adjusted to a reference point of each of regions of the subject placed on the cradle 12.

In step P2, a scout view is acquired. Scout views are usually acquired at zero degree and 90 degrees. Depending on a region such as the head, there is a case that only a scout view at 90 degrees is acquired. The details of acquisition of a scout view will be described later.

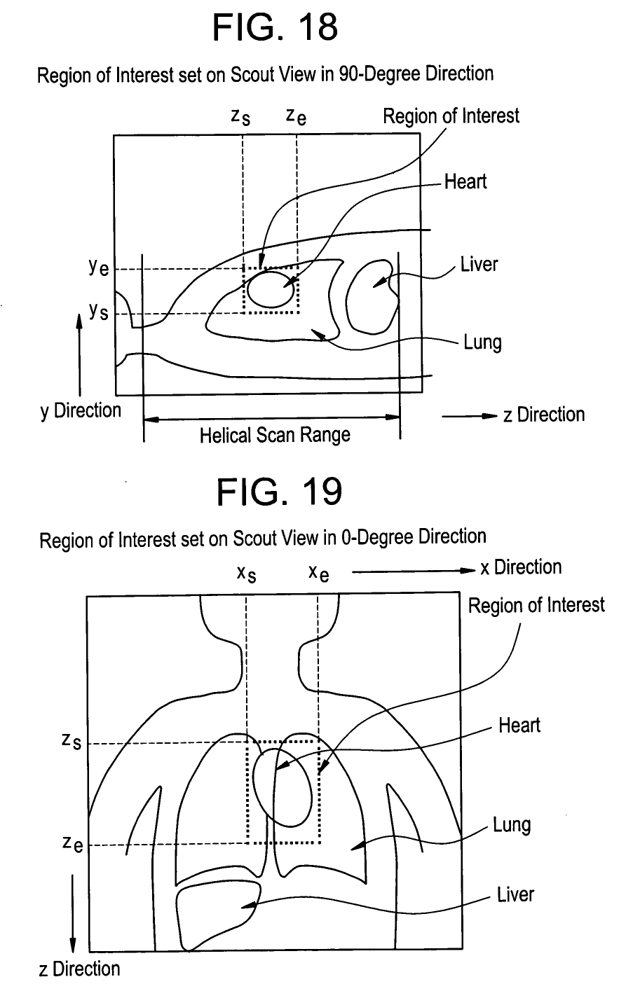

In step P3, image acquisition parameters are set. Usually, image acquisition is performed with the image acquisition parameters while displaying the position and size of a tomographic image on a scout view. In this case, the whole X-ray dose information of one helical scan, variable-pitch helical scan, conventional scan (axial scan), or cine scan is displayed and in addition, as shown in FIGS. 18 and 19, a region of interest is set on the scout view and X-ray dose information of the region of interest is displayed. In the cine scan, when the rotation speed or time is input, the X-ray dose information of the amount corresponding to the input rotation speed or the input time in the region of interest is displayed.

In step P4, a tomographic image is acquired. The details of acquisition of a tomographic image will be described later.

One example of obtaining information of dose of an X-ray applied to the subject will now be described.

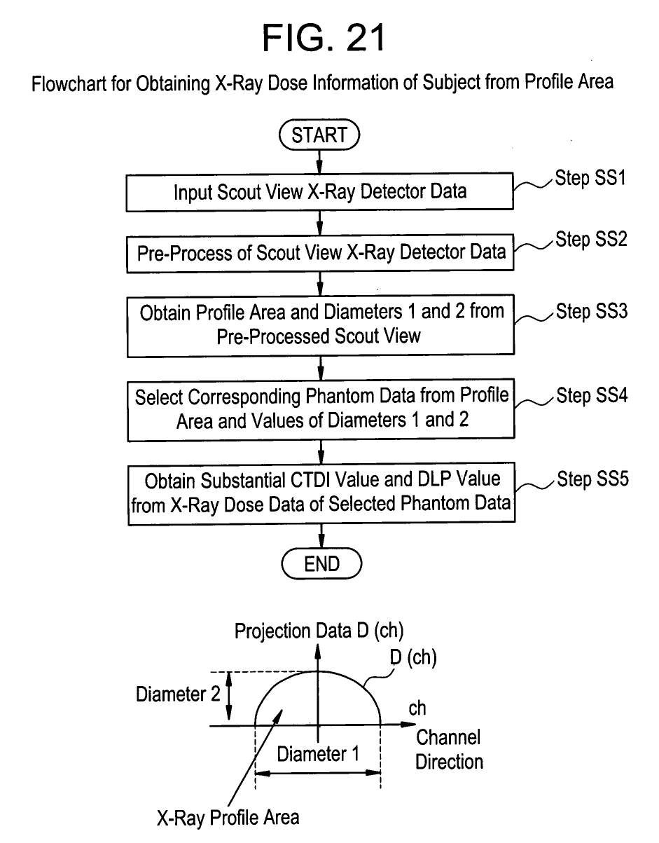

The distribution of dose of an X-ray applied to the subject is obtained on the basis of the size of the subject by the flow of processes as shown in FIG. 21.

In step SS1, scout view X-ray detector data is input.

In step SS2, the scout view X-ray detector data is pre-processed. The pre-process may be a process similar to the above-described pre-process of the scan.

In step SS3, a profile area and diameters 1 and 2 of the pre-processed scout view are obtained. The X-ray profile area Sx is sum of X-ray projection data values of all of the channels as shown by the following equation.

$${S}_{X} = {\sum }_{I = 1}^{CH} D ( i ) \qquad Equation 3$$

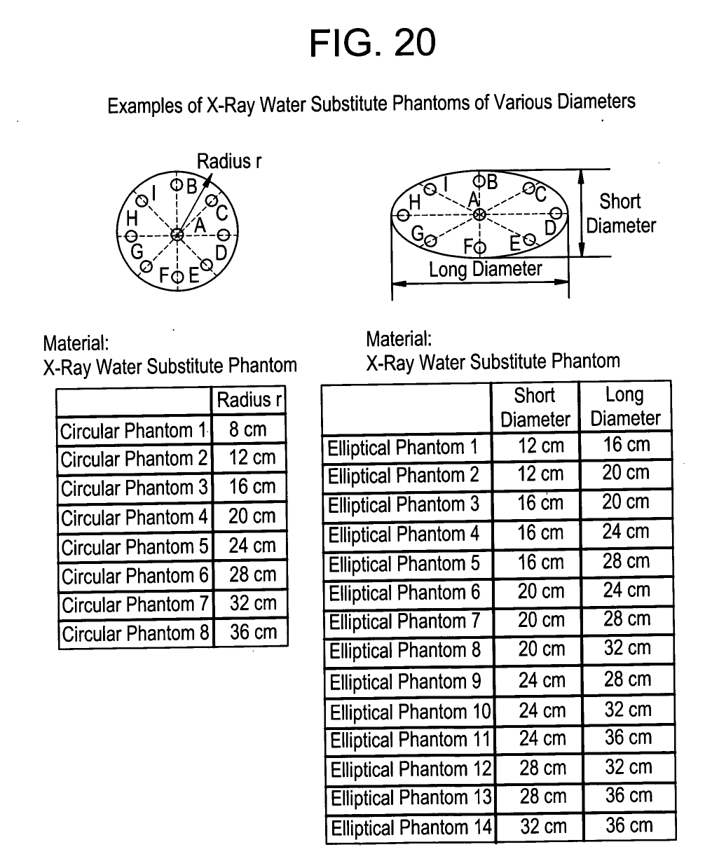

The correlation between the X-ray profile area Sx and a sectional area of a water substitute phantom shown in FIG. 20 is preliminarily held.

The length of the diameter 1 is a length R1 of continuous channels satisfying a threshold Th1 of noise level or larger, which is determined as follows.
Th1≦D(ch) Equation 4

From the number of the continuous channels, the length of projection in the x axis passing the center of the view of field (rotation center) or the y axis can be obtained from the intervals of channels of the X-ray detector and a geometric system of an X-ray data acquiring system.

For the diameter 2, projection data D(ch) is arranged in decreasing order of the value, that is, the decreasing order of X-ray absorption values. An average value of projection data of a certain number of channels, for example, 50 channels corresponding to 5% of all of the channels of, for example, 1,000 channels is obtained and converted to a length R2. The relation between the projection data value and the length of a water substitute material is preliminarily obtained by a conversion factor, a conversion table, or the like. A larger one of diameters 1R1 and 2R2 obtained as described above is set as a long diameter RL, and the shorter one is set as a short diameter RS.

In such a manner, the profile area Sx, the long diameter RL, and the short diameter RS are obtained.

In step SS4, corresponding phantom data is selected from the values of the profile area and the diameters 1 and 2. From the profile area Sx, the long diameter RL, and the short diameter RS obtained in step SS3, a CTDI value as X-ray dose information of the phantom of the water substitute material shown in FIG. 20 having the corresponding sectional area and long and short diameters is extracted. Alternately, a substantial CTDI value of a phantom having a similar size is extracted.

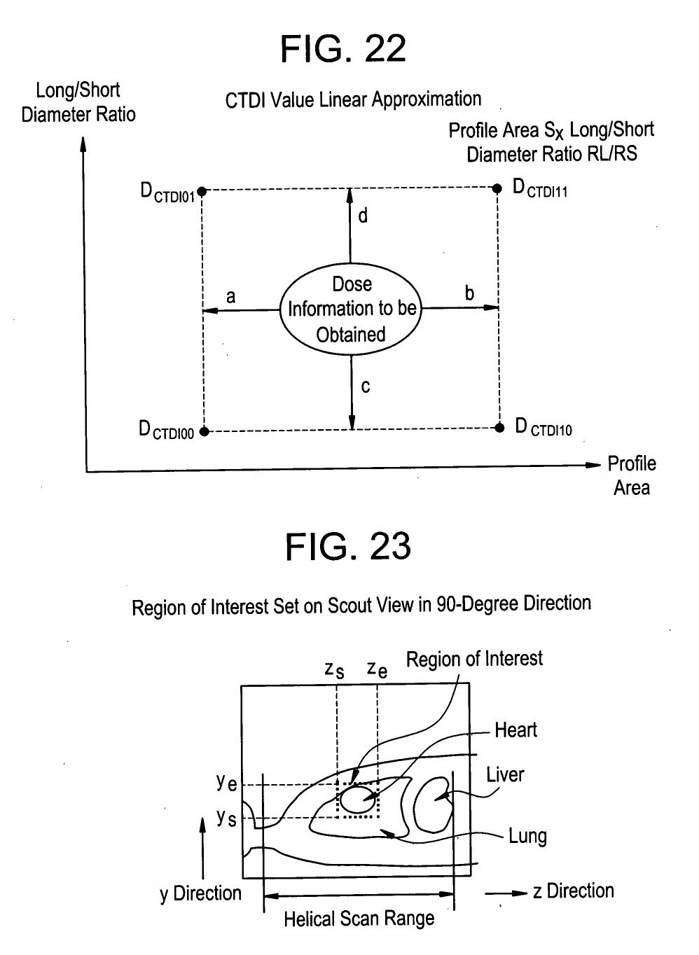

In step SS5, to obtain the substantial CTDI value and DLP value from the X-ray dose data of the selected phantom data, the extracted CTDI value is output as it is or a CTDI value in proximity is obtained by linear approximation. For example, as shown in FIG. 22, in the case of obtaining a CTDI value in the position of the profile area Sx and the ratio RL/RS of long and short diameters, by setting CTDI values in close four points as D_{CTDIS1}, D_{CTDIS2}, D_{CTDIR1}, and D_{CTDIR2} and setting parameter distances to the points as a, b, c, and d, the CTDI value D_{CTDI} of dose information to be obtained is derived by the following.

$${D}_{CTDI} = \frac{d}{c + d} ( \frac{b}{a + b} \cdot {D}_{CTDI 00} + \frac{a}{a + b} \cdot {D}_{CTDI 10} ) + \frac{c}{c + d} ( \frac{b}{a + b} \cdot {D}_{CTDI 01} + \frac{a}{a + b} \cdot {D}_{CTDI 11} ) \qquad Equation 5$$

The DLP value is obtained from the CTDI value.

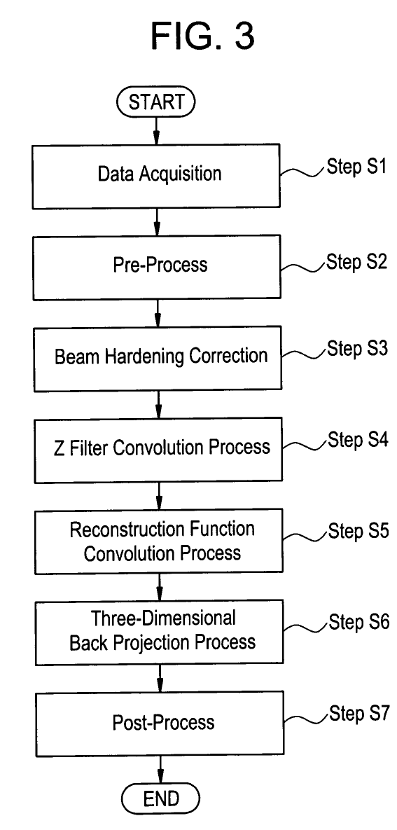

FIG. 3 is a flowchart showing an outline of operations of acquiring a tomographic image and a scout view of the X-ray CT apparatus 100 of the present invention.

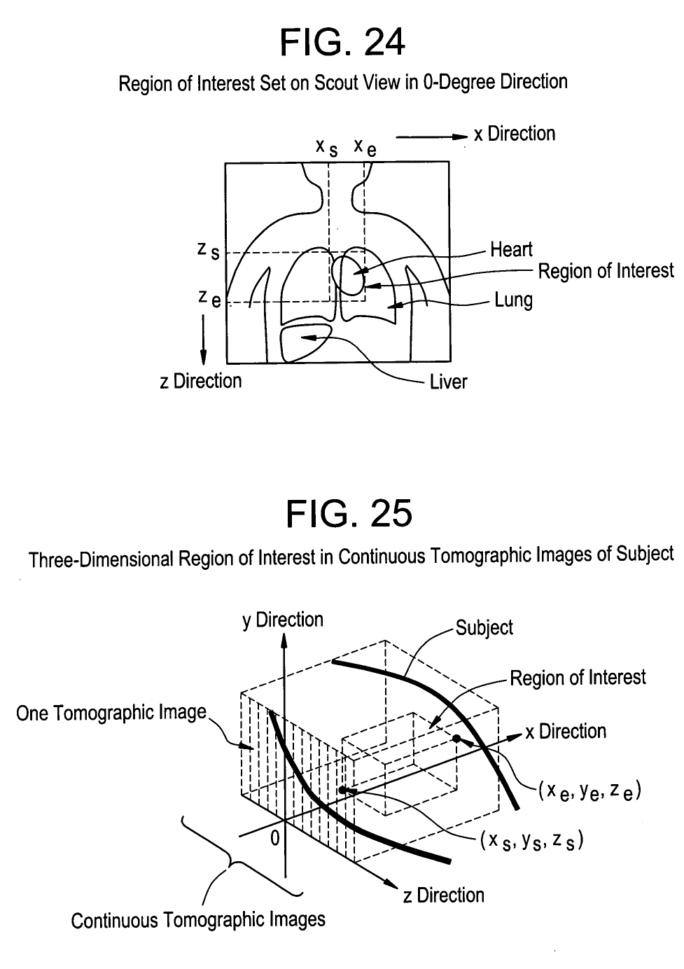

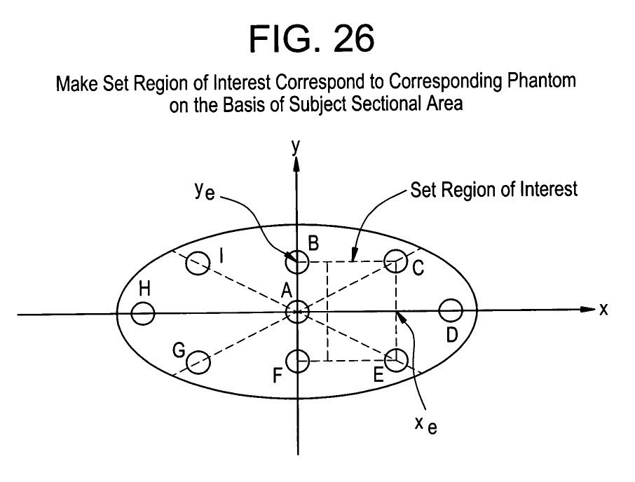

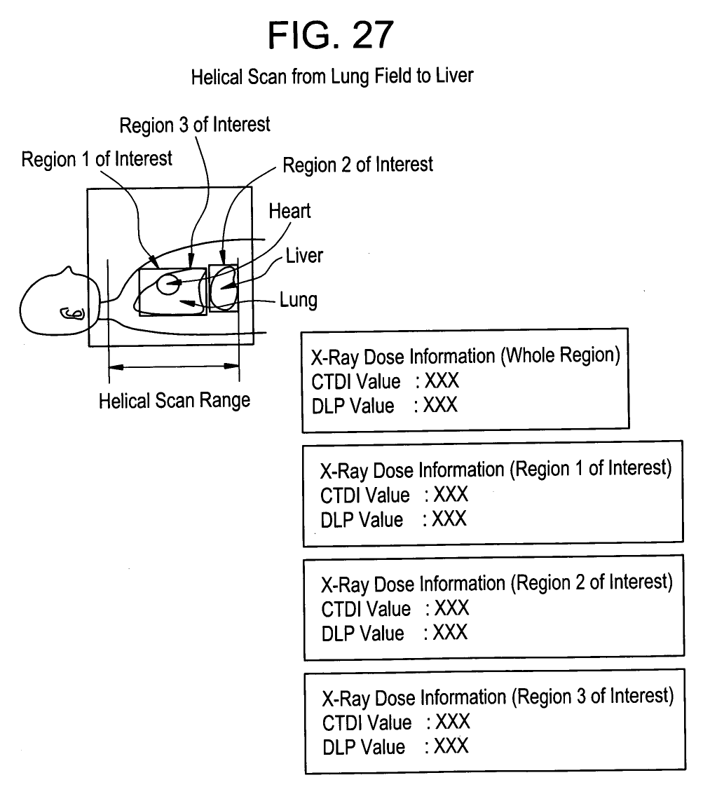

In the following, the case of the multi-row X-ray detector 24 will be described but the case of the two-dimensional X-ray area detector 24 having a matrix structure typified by a flat panel X-ray detector is similar. In the case of obtaining a CTDI value of only a three-dimensional region of interest in tomographic images continuous in the z direction as shown in FIG. 23, start and end points (Zs, Ze) in the z-direction coordinate and start and end points (Ys, Ye) in the y-direction coordinate are determined on a scout view of the 90-degree direction. As shown in FIG. 24, start and end points (Xs, Xe) in the x-direction coordinate are determined on a scout view of the 0-degree direction. In such a manner, a three-dimensional region of interest can be set on a subject from two directions of the scout view in the 0-degree direction and the scout view in the 90-degree direction as shown in FIG. 25. The set region of interest is transferred to a phantom equivalent to each tomographic image as shown in FIG. 26. The X-ray dose information in each of points in the region of interest set in FIG. 27 is obtained by linear approximation on the basis of the X-ray dose information D_{CTDIA} in the center position and the X-ray dose information D_{CTDIB}, D_{CTDIC}, D_{CTDID}, D_{CTDIE}, D_{CTDIF}, D_{CTDIG}, D_{CTDIH}, and D_{CTDII} in eight peripheral positions.

In step S1, in a helical scan, while rotating the X-ray tube 21 and the multi-row X-ray detector 24 around the subject and moving the cradle 12 on the image acquisition table 10 linearly, X-ray detector data is acquired. The X-ray detector data is acquired by adding a table linear movement z-direction position Ztable(view) to X-ray detector data D0 (view, j,i) expressed by a view angle “view”, a detector column number “j”, and a channel number “i”. In a variable-pitch helical scan, data is acquired not only at constant speed but also at the time of acceleration and deceleration in a helical scan.

In the conventional scan (axial scan) or cine scan, while fixing the cradle 12 on the image acquisition table 10 in a position in the z direction, a data acquiring system is allowed to revolve once or a plurality of times to acquire X-ray detector data. As necessary, after the cradle 12 is moved to the next position in the z direction, the data acquiring system is allowed to revolve again once or a plurality of times to acquire X-ray detector data.

In the scout view acquisition, the X-ray tube 21 and the multi-row X-ray detector 24 are fixed and the X-ray detector data is acquired while the cradle 12 on the image acquisition table 10 is moved linearly.

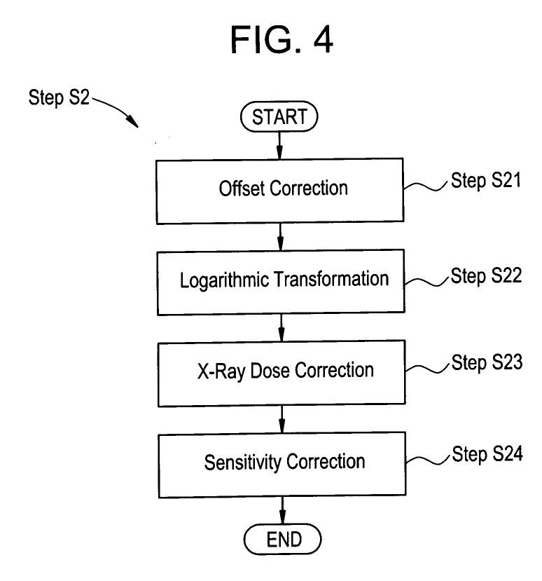

In step S2, the X-ray detector data D0 (view, j, i) is converted to projection data by a pre-process. The pre-process includes, as shown in FIG. 4, offset correction in step S21, logarithmic transformation in step S22, X-ray dose correction in step S23, and sensitivity correction in step S24.

In the case of scout view acquisition, a scout view is completed by displaying the pre-processed X-ray detector data while adjusting the pixel size in the channel direction and the pixel size in the z direction as the cradle linear movement direction to the display pixel size of the monitor 6.

In step S3, beam hardening correction is made on the pre-processed projection data D1 (view, j, i). When the projection data subjected to the sensitivity correction S24 in the pre-process S2 is set as D1(view, j, i) and the data subjected to the beam hardening correction S3 is set as DI1 (view, j, i), the beam hardening correction S3 is expressed, for example, by a polynomial form.
D11(view,j,i)=D1(view,j,i)·(Bo(j,i)+B₁(j,i)·D1(view,j,i)+B₂(j,i)·D1(view,j,i)²) Equation 6

Since the independent beam hardening correction can be made every j detectors, if the tube voltages of the data acquisition systems are different from each other with the image acquisition parameters, variations in the X-ray energy characteristics among detectors can be corrected.

In step S4, z-filter convolution process for applying z-direction (column direction) filtering to the projection data D11 (view, j, i) subjected to the beam hardening correction is performed.

In step S4, after the pre-process in each view angle and each data acquiring system, filtering whose filter size in the column direction is five columns is performed on projection data of the multi-row X-ray detector D11 (view, j, i) (i=1 to CH,j=1 to ROW), which has been subjected to the beam hardening correction.

(w₁(j), w₂(j), w₃(j), w₄(j), w₅(j))

where

$${\sum }_{k = 1}^{5} {w}_{k} ( j ) = 1 \qquad Equation 7$$

The corrected detector data D12 (view, j, i) is expressed as follows.

$$D 12 ( view , j , i ) = {\sum }_{k = 1}^{5} ( D 11 ( view , j - k - 3 , i ) \cdot {w}_{k} ( j ) ) \qquad Equation 8$$

When the maximum number of channels is CH and the maximum number of columns is ROW, the following is obtained.
D11(view,−1,i)=D1(view,0,i)=D11(view,1,i)
D11(view,ROW,i)=D11(view, ROW+1,i)=D11(view, ROW+2,i) Equation 9

By changing the column-direction filter factor every channel, the slice thickness can be controlled according to the distance from the center of image reconstruction. In a tomographic image, the peripheral portion is generally thicker than the reconstruction center. Consequently, by making the column-direction filter factor in the center portion and that in the peripheral portion different from each other so that the column-direction filter factor changes in a wide range near the center channel and changes in a narrow range near the peripheral channels, the slice thickness can be uniform in the peripheral and center portions in image reconstruction.

By controlling the column-direction filter factors in the center channel and the peripheral channel of the multi-row X-ray detector 24, the slice thickness can be controlled in each of the center portion and the peripheral portion. By slightly increasing the slice thickness with the column-direction filter, artifact and noise are largely reduced. In such a manner, the degree of reducing artifact and the degree of reducing noise can be also controlled. In other words, the quality of a tomographic image reconstructed as a three-dimensional image, that is, an xy plane can be controlled. As another embodiment, by using a deconvolution filter as a column-direction (z-direction) filter factor, a tomographic image of thin slice thickness can be also realized.

In step S5, reconstruction function convolution process is performed. Specifically, data is subjected to Fourier transform and the resultant data is multiplied with a reconstruction function and is subjected to inverse Fourier transform. In the reconstruction function convolution process S5, when data subjected to the z filter convolution process is set as D12, data subjected to the reconstruction function convolution process is set as D13, and a reconstruction function to be convoluted is set as Kernel (j), the reconstruction function convolution process is expressed as follows.
D13(view,j,i)=D12(view,j,i)*Kernel(j) Equation 10

That is, an independent reconstruction function convolution process can be performed every j detectors with the reconstruction function kernel (j), so that variations in the noise characteristic and resolution characteristic can be corrected on the column unit basis.

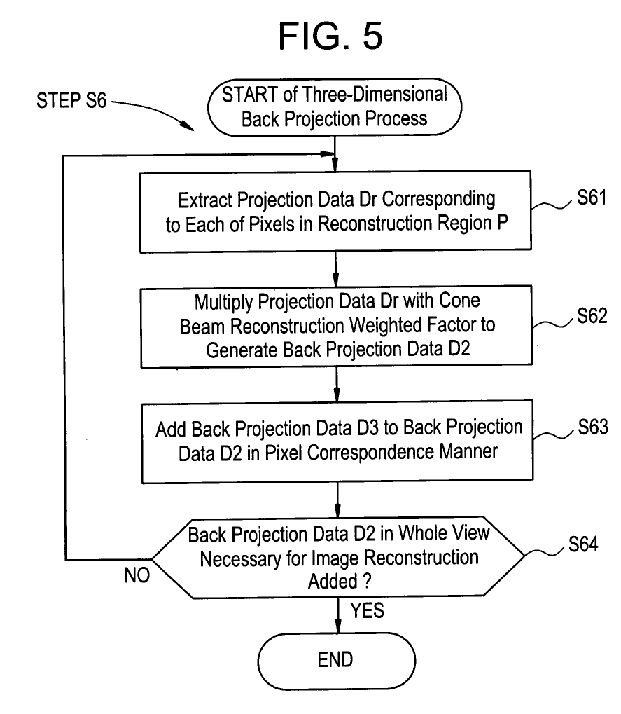

In step S6, three-dimensional back projection process is performed on the projection data D13 (view, j, i) subjected to the reconstruction function convolution process, thereby obtaining back projection data D3 (x, y). An image to be reconstructed is reconstructed to a three-dimensional image in an xy plane as a plane perpendicular to the z axis. It is assumed that the following reconstruction region P is parallel to the xy plane. The three-dimensional back projection process will be described later with reference to FIG. 5.

In step S7, post processes such as image filter convolution and CT value conversion are performed on the back projection data D3 (x, y, z), thereby obtaining a tomographic image D31 (x, y).

In the image filter convolution process in the post-process, when the tomographic image subjected to the three-dimensional back projection is set as D31 (x, y, z), the data subjected to the image filter convolution is set as D32 (x, y, z), and the image filter is set as Filter(z), the following expression is obtained.
D32(x,y,z)=D31(x,y,z)*Filter(z) Equation 11

Since the independent image filter convolution process can be performed every j detectors, variations in the noise characteristics and resolution characteristic can be corrected every j detectors.

Acquired tomographic images are displayed on the monitor 6.

FIG. 5 is a flowchart showing the details of the three-dimensional back projection process (step S6 in FIG. 4).

In the embodiment, an image is reconstructed as a three-dimensional image in a plane perpendicular to the z axis, that is, an xy plane. In the following, it is assumed that the reconstruction region P is parallel to the xy plane.

In step S61, attention is paid to one of all of views necessary for reconstructing a tomographic image (that is, view of 360 degrees or a view of “180 degrees+the amount of the fan angle”) and projection data Dr corresponding to each of pixels in the reconstruction region P is extracted.

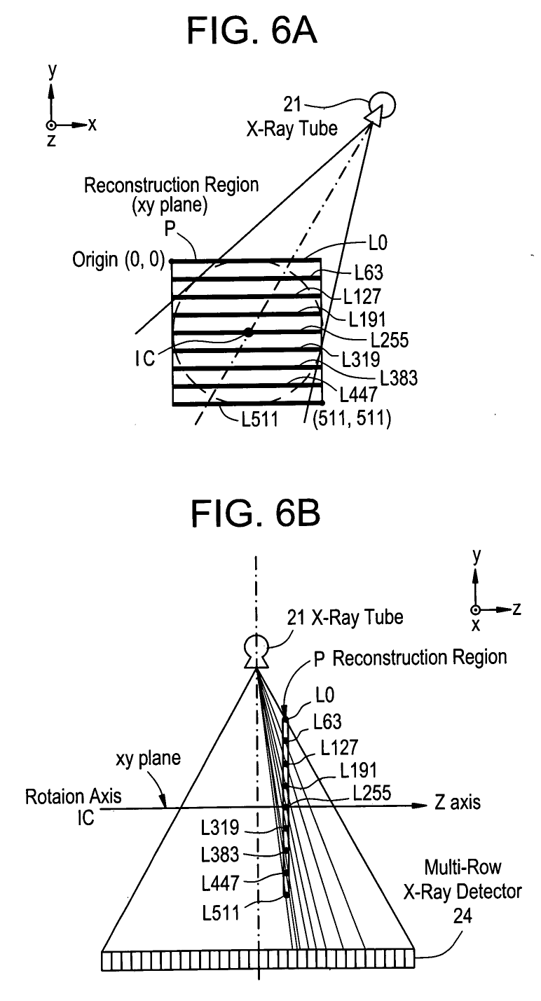

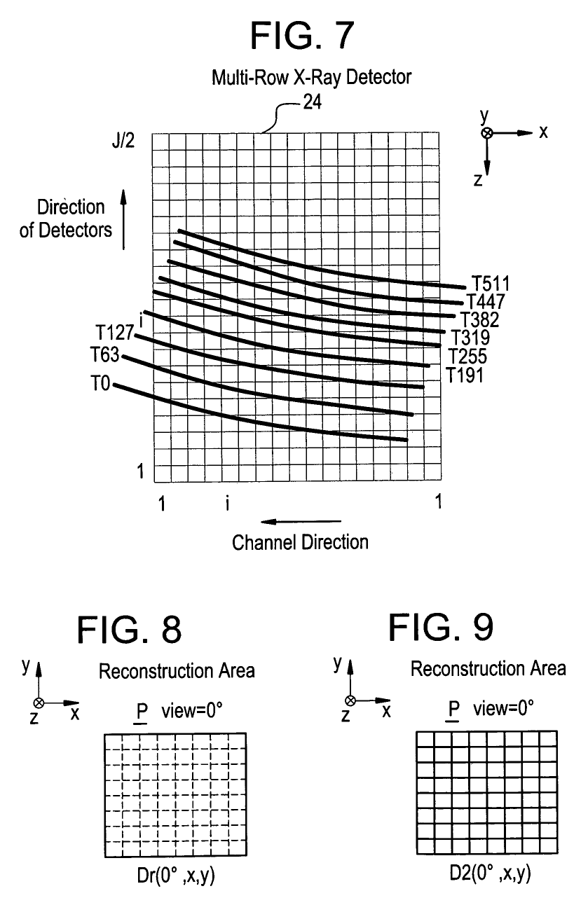

As shown in FIGS. 6A and 6B, a square region of 512×512 pixels parallel to the xy plane is set as the reconstruction region P, and a pixel line L0 parallel to the x axis at y=0, a pixel line L63 at y=63, a pixel line L127 at y=127, a pixel line L191 at y=191, a pixel line L255 at y=255, a pixel line L319 at y=319, a pixel line L383 at y=383, a pixel line L447 at y=447, and a pixel line L511 at y=511 are set as lines. Projection data on lines T0 to T511 as shown in FIG. 7 obtained by projecting the pixel lines L0 to L511 onto the plane of the multi-row X-ray detector 24 in an X-ray transmission direction is extracted as projection data Dr (view, x, y) of the pixel lines L0 to L511, “x, y” in Dr (view, x, y) corresponds to each pixel (x, y) in a tomographic image.

The X-ray transmission direction is determined by geometric positions of an X-ray focal point of the X-ray tube 21, the pixels, and the multi-row X-ray detector 24. Since the z coordinate z (view) of the X-ray detector data D0 (view, j, i) is attached as table linear movement z direction position Ztable (view) to the X-ray detector data and is known, the X-ray focal point and the X-ray transmission direction in a data acquisition geometric system of a multi-row X-ray detector can be accurately obtained with X-ray detector data D0 (view, j, i) during acceleration/deceleration.

In the case where, for example, part of a line is out in the channel direction of the multi-row X-ray detector 24 like the line T0 obtained by projecting the pixel line L0 to the plane of the multi-row X-ray detector 24 in the X-ray transmission direction, corresponding projection data Dr (view, x, y) is set to “0”. In the case where a line is out in the z direction, projection data Dr (view, x, y) is obtained by extrapolation.

In such a manner, as shown in FIG. 8, the projection data Dr (view, x, y) corresponding to each of pixels of the reconstruction region P can be extracted.

Referring again to FIG. 5, in step S62, the projection data Dr (view, x, y) is multiplied with a cone beam reconstruction weighted factor, thereby generating projection data D2 (view, x, y) as shown in FIG. 9.

The cone beam reconstruction weighted factor w (i, j) is as follows. In the case of fan beam image reconstruction, generally, when the angle formed by a straight line connecting the focal point of the X-ray tube 21 at view=βa and a pixel g (x, y) on the reconstruction region P (xy plane) and the center axis Bc of an X-ray beam is set as γ and an opposed view is set as view=βb, the following expression is obtained.
βb=βa+180°−2γ Equation 12

When the angle formed by an X-ray beam passing the pixel g (x, y) on the reconstruction region P and the reconstruction plane P is αa and the angle formed by an X-ray beam opposite to the X-ray beam passing the pixel g (x, y) and the reconstruction plane P is αb, the angles αa and αb are multiplied with the dependent cone beam reconstruction weighted factors ωa and ωb and the resultants are added, thereby obtaining back projection pixel data D2 (0, x, y).
D2(0,x,y)=ωa·D2(0,x,y)_{—}a+ωb·D2(0,x,y)_{—}b Equation 13

where D2 (0, x, y)_a denotes projection data of a view βa, and D2 (0, x, y)_b denotes projection data of a view βb.

The sum of the opposed beams of the cone beam reconstruction weighted factors is obtained as follows.
ωa+ωb=1 Equation 14

By multiplying the projection data with the cone beam reconstruction weighted factors ωa and ωb and adding the resultants, cone angle artifact can be reduced.

For example, the cone beam reconstruction weighted factors ωa and ωb obtained by the following equations can be used. Further, ga denotes a weighted factor of an X-ray beam, and gb denotes a weighted factor of the opposed X-ray beam.

When the half of a fan beam angle is γmax, the following is obtained.
ga=f(γmax,αa,βa)
ga=f(γmax,αb,βb)
xa=2·ga^{q}/(ga^{q}+gb^{q})
xb=2·gb^{q}/(ga^{q}+gb^{q})
wa=xa²·(3−2xa)
wb=xb²·(3−2xb) Equation 15

(For example, q is set to 1.)

For example, as an example of ga and gb, max[ ] is a function employing a larger value, and the following is obtained.
ga=max[0,{(π/2+γmax)−|βa|}]·|tan(aa)|
gb=max[0,{(π/2+γmax)−|βb|}]·|tan(ab)| Equation 16

In the case of fan beam image reconstruction, each of the pixels on the reconstruction region P is multiplied with a distance factor. When the distance from the focal point of the X-ray tube 21 to the detector “j” of the multi-row X-ray detector 24 corresponding to the projection data Dr and the channel “i” is set as r0 and the distance from the focal point of the X-ray tube 21 to a pixel on the reconstruction region P corresponding to the projection data Dr is set as r1, the distance factor is (r1/r0)².

In the case of parallel beam image reconstruction, it is sufficient to multiply each of pixels in the reconstruction region P only with a cone beam reconstruction weighted factor w (i, j).

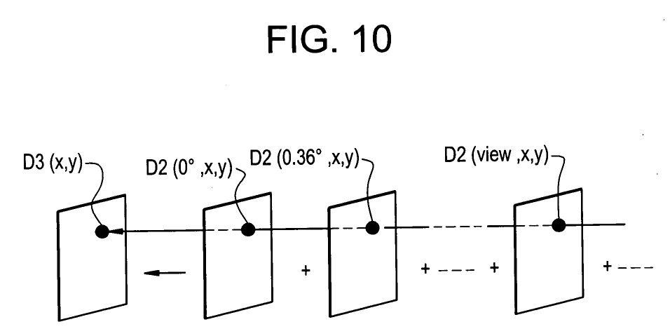

In step S63, as shown in FIG. 10, the projection data D2 (view, x, y) is added to back projection data D3 (x, y) which is preliminarily cleared on a pixel-to-pixel correspondence manner.

In step S64, steps S61 to S63 are repeated on all of the views necessary to reconstruct a tomographic image (that is, a view of 360 degrees or a view of “180 degrees+the amount of fan degree”), thereby obtaining back projection data D3 (x, y) as shown in FIG. 10.

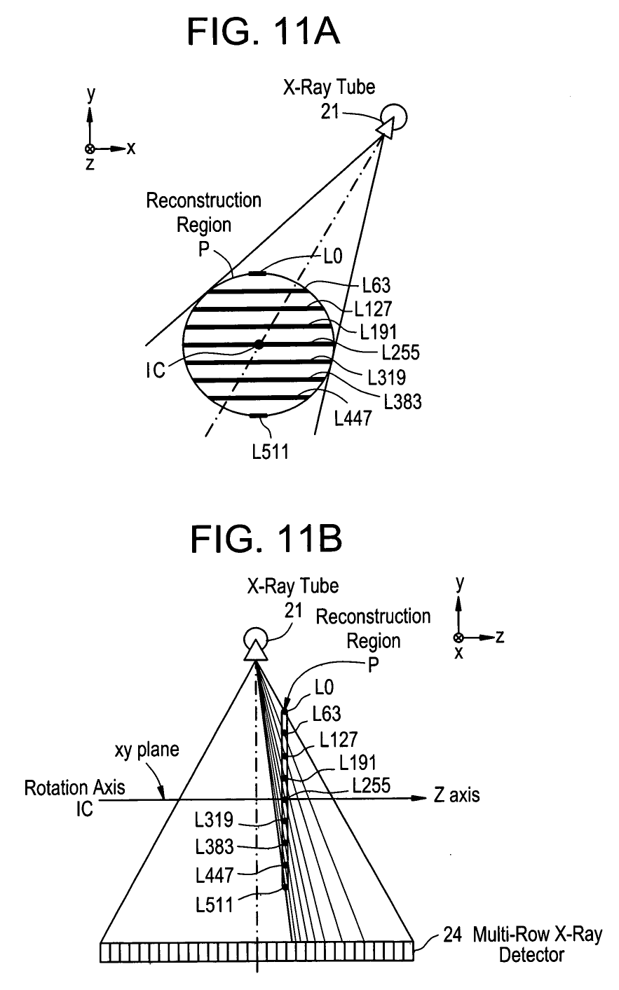

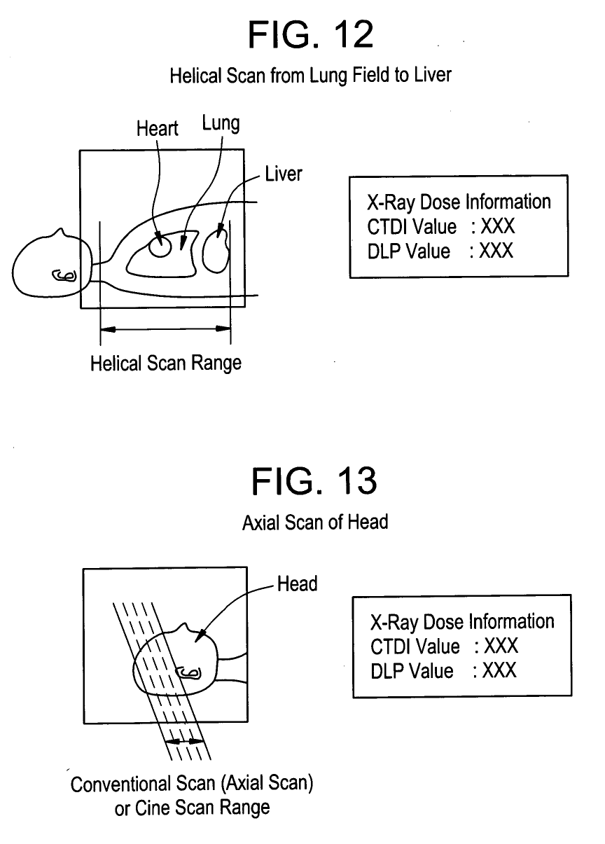

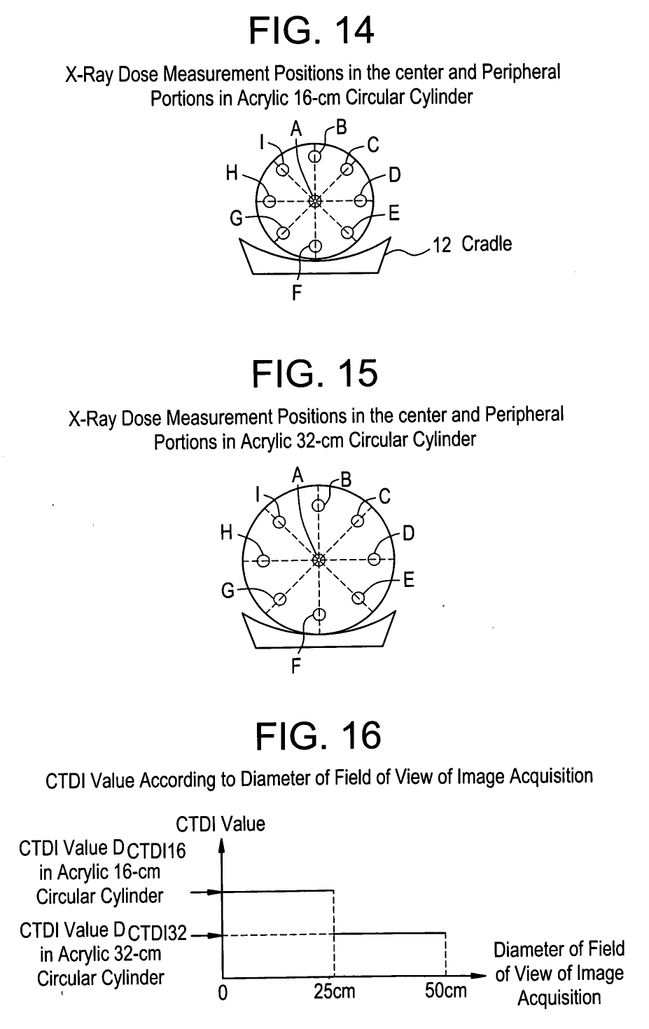

The reconstruction region P is not limited to the square region of 512×512 pixels but may be a circular region having a diameter of 512 pixels as shown in FIGS. 11A and 11B.

### EXAMPLE 1

When the embodiment is applied to an actual helical scan, X-ray dose information of the whole region of image acquisition, X-ray does information of a region 1 of interest (heart), and X-ray dose information of a region 2 of interest (liver) is known. In view of sensitivity to X-ray exposure of each of the organs, reduction in the exposure of the subject can be considered.

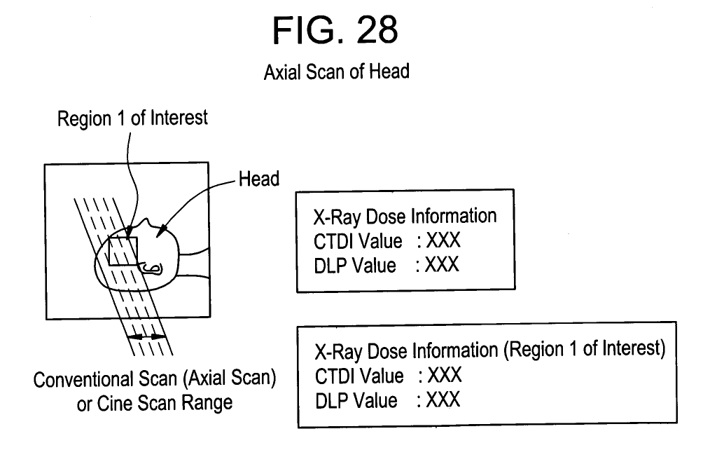

Also in a conventional scan (axial scan) or a cine scan, similarly, each of the X-ray dose information of the whole region of image acquisition and X-ray dose information of the region 1 of interest is known as shown in FIG. 28, so that the X-ray exposure of each of the organs and the X-ray exposure of the whole region can be taken into consideration.

### EXAMPLE 2

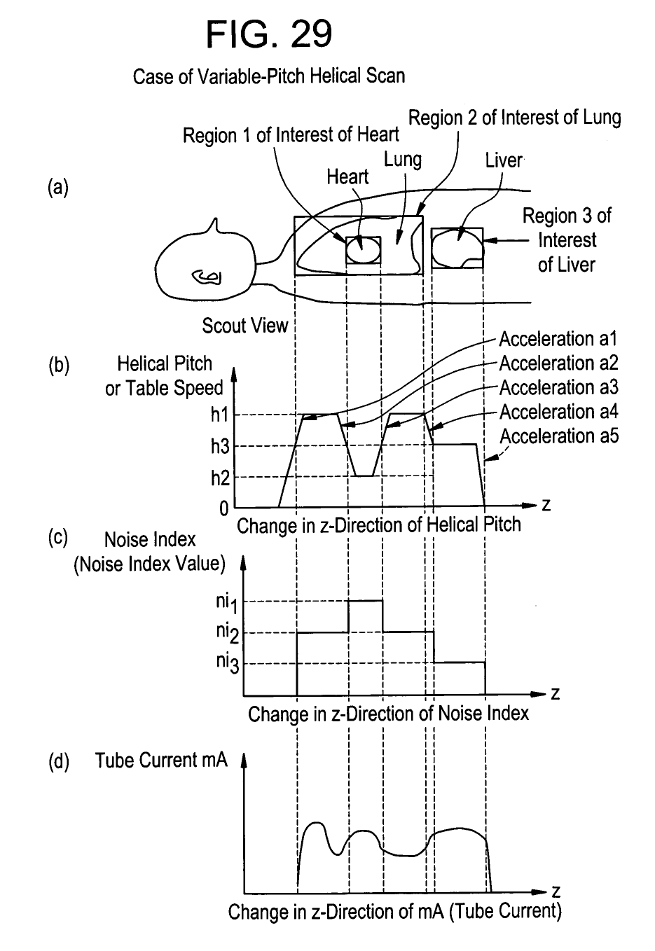

In Example 2, the case of a variable-pitch helical scan as shown in FIG. 29 will be described. In the variable-pitch helical scan, as shown in FIG. 29, the helical pitch and noise index (index value of image noise) vary in the z-direction range, for example, in the heart, liver, and lung field. Consequently, the X-ray dose information in the positions in the z-direction is not easily known at a glance in comparison with a normal conventional scan (axial scan), a cine scan, or a helical scan, so that it is even more necessary to display the X-ray dose information. In this case as well, by displaying the X-ray dose information with respect to each of the whole region, the region 1 of interest (heart), the region 2 of interest (lung field), and the region 3 of field (liver), the information is shown more clearly to the operator. Therefore, reduction in the exposure of the subject can be considered in view of the sensitivity to X-ray exposure of each of the organs.

### EXAMPLE 3

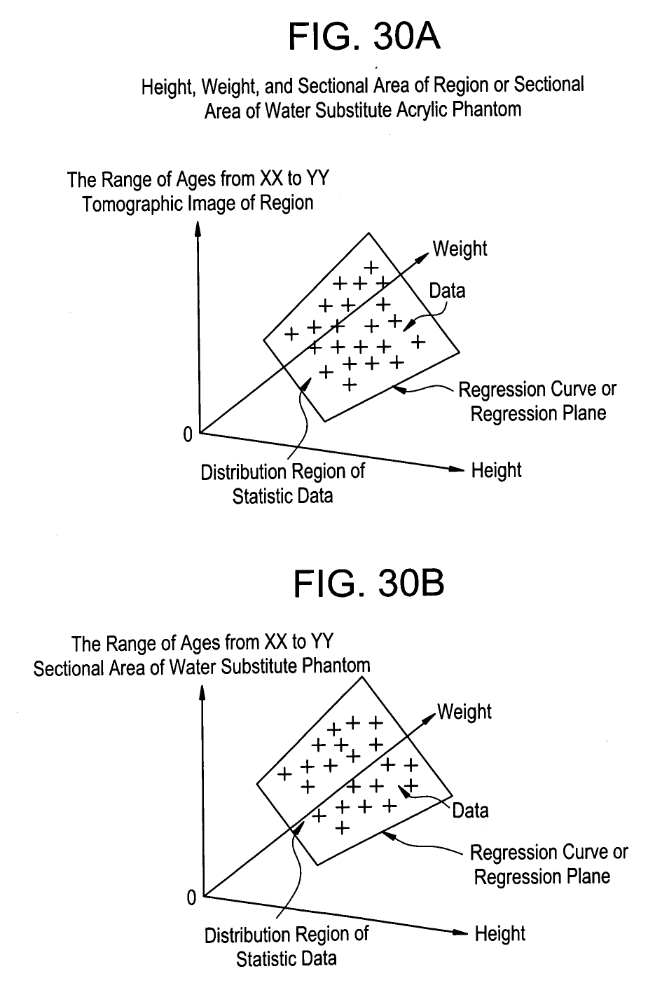

In Example 3, an X-ray profile area Sx obtained from a scout view is used to obtain the correlation with a water substitute phantom to be referred to. Height, weight, age, an image acquisition region, and sex are investigated statistically. As shown in FIG. 30A, the relations among weight, height, and sectional area of a region are obtained with respect to each of sex, the range of ages, and regions, and a regression plane or regression curve is derived from distributed statistic data. Alternately, as shown in FIG. 30B, the relations among the weight, height, and sectional area of a water substitute phantom are obtained, and a regression plane or regression curve is derived from distributed statistic data. An expression of the regression plane or regression curve is also obtained.

When sex, age, a region, weight, and height are entered, the sectional area of the region and the sectional area of a water substitute phantom are obtained by the expression of the regression plane or regression curve. The water substitute phantom to be referred to is determined, and X-ray dose information is determined. When a region of interest is set, X-ray dose information in the region of interest is obtained.

According to the X-ray CT apparatus or X-ray CT imaging method of the present invention, the X-ray CT apparatus 100 produces an effect of reducing exposure in a conventional scan (axial scan), a cine scan, or a helical scan in X-ray cone beams extending in the z direction existing at the start and end of the conventional scan (axial scan), the cine scan, or the helical scan of the X-ray CT apparatus having a multi-row X-ray detector or a two-dimensional area X-ray detector of a matrix structure typified by a flat panel X-ray detector.

### INDUSTRIAL APPLICABILITY

As the image reconstruction method in the embodiments, a three-dimensional image reconstruction method by a conventionally known feldkamp reconstruction may be employed. Further, another three-dimensional image reconstruction may be also employed. Alternately, a two-dimensional image reconstruction may be employed.

Although the X-ray CT apparatus having a multi-row X-ray detector or a two-dimensional area X-ray detector of a matrix structure typified by a flat panel X-ray detector has been described in the embodiment, similar effects can be also produced by an X-ray CT apparatus of a single X-ray detector.

In the embodiment, column-direction (z-direction) filters of different factors are convoluted, thereby realizing adjustment of variations in picture quality, and picture quality with uniform slice thickness, artifact, and noise among the columns. Various filter factors can be employed and similar effects can be produced by using any of the various filter factors.

Although the X-ray CT apparatus for medical use has been described in the foregoing embodiment, the invention can be also applied to an industrial X-ray CT apparatus, an X-ray CT-PET apparatus and an X-ray CT-SPECT apparatus combined with another apparatus, and so on.

Although X-ray water substitute phantoms of circular and elliptic shapes having various diameters are used in the embodiment as shown in FIG. 20, similar effects can be expected with other shapes and other materials.

In the embodiment, the X-ray dose information in each of points of the regions of interest which are set as shown in FIG. 26 is obtained by linear approximation between the center position A of the phantom and the peripheral positions B to I of the phantom, and the total of the points is used as the X-ray dose information of the region of interest. Similar effects can be expected when the X-ray dose information is obtained by other calculating methods. For example, also in the case of roughly correcting and obtaining X-ray dose information of a phantom equivalent to a section of a subject with the area and position of the region of interest, similar effects can be expected.

1. An X-ray CT apparatus comprising:
a device for acquiring projection data of an X-ray passed through a subject positioned between an X-ray generator and an X-ray detector which are opposite to each other;
a device for reconstructing an image from the projection data acquired by said device for acquiring the projection data;
a device for displaying a tomographic image obtained by said device for reconstructing the image;
a setting device for setting various image acquisition parameters for acquisition of a tomographic image; and
a device for displaying X-ray dose information of a partial region of an image acquisition region provided by one scan when an image acquisition parameter setting process is executed.

2. An X-ray CT apparatus according to claim 1, wherein said X-ray detector is any one of a matrix structure two-dimensional X-ray area detector, a flat panel X-ray detector, and a multi X-ray detector.

3. An X-ray CT apparatus according to claim 1, wherein said device for displaying the X-ray dose information includes a device for displaying X-ray dose information of a partial region of an image acquisition region provided by one scan in a z-direction as a direction of a body axis of the subject when an image acquisition parameter setting process of a conventional scan or an axial scan is executed.

4. An X-ray CT apparatus according to claim 1, wherein said device for displaying the X-ray dose information includes a device for displaying X-ray dose information of a partial region of an image acquisition region provided by one scan in a z-direction as a direction of a body axis of the subject when an image acquisition parameter setting process of a helical scan or a variable-pitch helical scan is executed.

5. An X-ray CT apparatus according to claim 1, wherein said device for displaying the X-ray dose information includes a device for displaying X-ray dose information of a partial region of an image acquisition region provided by one scan in a z-direction as a direction of a body axis of the subject or in a time direction when an image acquisition parameter setting process of a cine scan is executed.

6. An X-ray CT apparatus according to claim 1, wherein said partial region is being set on a scout view of the subject.

7. An X-ray CT apparatus according to claim 1, wherein said partial region is a region of interest and being set by setting a part of one scan range in a z-direction and, in a case where a vertical direction perpendicular to the z-direction is set as a y-direction and a direction perpendicular to the z-direction and the y-direction is set as an x-direction, designating a range in at least one of the x-direction and the y-direction.

8. An X-ray CT apparatus according to claim 1, wherein said X-ray dose information includes at least one of a CTDI value, a DLP value, and efficiency for X-ray utilization.

9. An X-ray CT apparatus according to claim 1, wherein said X-ray dose information includes a value depending on a sectional area of the subject or an X-ray profile area obtained from a scout view of the subject.

10. An X-ray CT apparatus according to claim 9, wherein said sectional area is predicted from at least one of height, weight, age, an image acquisition part, and sex of the subject.

11. An X-ray CT apparatus according to claim 9, wherein said sectional area is predicted from the X-ray profile area.
